(19) United States
(12) Patent Application Publication
(10) Pub. No.: US 2013/0231365 A1
(43) Pub. Date: Sep. 5, 2013

(54) Treatment of Inflammation with Certain Alpha-7 Nicotinic Acid Receptor Agonists in Combination with Acetylcholinesterase Inhibitors

(71) Applicant: Gerhard Koenig, Newton, MA (US)

(72) Inventor: Gerhard Koenig, Newton, MA (US)

(21) Appl. No.: 13/988,205

(22) PCT Filed: Nov. 18, 2011
(86) PCT No.: PCT/US2011/061519

§ 371 Date: May 17, 2013

Related U.S. Application Data
(60) Provisional application No. 61/415,294, filed on Nov. 18, 2010.

(51) Int. Cl. A61K 31/439; A61K 45/06

(52) U.S. Cl. CPC A61K 31/439; A61K 45/06
USPC 514/305

(57) ABSTRACT

A method for treating inflammation comprising administering to a patient (R)-7-chloro-N-(quinuclidin-3-yl)benzo[b]thiophene-2-carboxamide or a pharmaceutically acceptable salt thereof or (R)-7-(2-methoxyphenyl)-N-(quinuclidin-3-yl)benzofuran-2-carboxamide or certain other alpha 7 receptor agonists combination with an acetylcholinesterase inhibitor.

### BACKGROUND

Nicotinic acetylcholine receptors (nAChR) form a family of ion channels activated by acetylcholine. Functional receptors typically contain five subunits and there are numerous receptor subtypes. Studies have shown that central nicotinic acetylcholine receptors are involved in learning and memory. Nicotinic acetylcholine receptors of the alpha7 subtype are prevalent in the hippocampus and cerebral cortex.

Certain agonists of the alpha7 nAChR are known to inhibit inflammatory cytokine production and are thought to be useful in treating inflammation (Rosas-Ballina et al. 2009 Molecular Medicine 15:195-2002). Such agonists are thought to act by reduce release of certain pro-inflammatory factors such as TNF.

WO 2003/055878 describes a variety of agonists of the alpha7 nAChR said to be useful for improving cognition. Among the compounds described is (R)-7-chloro-N-(quinuclidin-3-yl)benzo[b]thiophene-2-carboxamide.

At least four drugs that have been used to treat AD, tacrine (1,2,3,4-tetrahydroacridin-9-amine), donepezil (donepezil HCl; 1-benyzl-4-[(5,6-dimethoxy-1-indanon)-2-yl]methylpiperidine monohydrochloride), rivastigmine ((S)—N-Ethyl-N-methyl-3-[1-(dimethylamino)ethyl]-phenyl carbamate) and galantamine (galantamine hydrobromide; (4aS,6R,8aS)-4a,5,9,10,11,12-hexahydro-3-methoxy-11-methyl-6H-benzofuro[3a,3,2-ef][2]benzazepin-6-ol hydrobromide), appear to act as acetylcholinesterase inhibitors that increase acetylcholine in the CNS.

### SUMMARY

Described herein are methods for treating various types of inflammation with certain alpha 7 nicotinic acid receptor agonists, for example, (R)-7-chloro-N-(quinuclidin-3-yl)benzo[b]thiophene-2-carboxamide or a pharmaceutically acceptable salt thereof (e.g., (R)-7-chloro-N-(quinuclidin-3-yl)benzo[b]thiophene-2-carboxamide hydrochloride monohydrate) or (R)-7-(2-methoxyphenyl)-N-(quinuclidin-3-yl)benzofuran-2-carboxamide (N-[(3R)-1-azabicyclo[2.2.2]oct-3-yl]-7-[2-(methoxy)phenyl]-1-benzofuran-2-carboxamide) or a pharmaceutically acceptable salt thereof (e.g., R)-7-(2-methoxyphenyl)-N-(quinuclidin-3-yl)benzofuran-2-carboxamide hydrochloride) or a compound of Formula I or a pharmaceutically acceptable salt thereof in combination with an acetylcholinesterase inhibitor (e.g., donepezil or rivastigmine).

In some embodiments the methods provide a therapeutic benefit when both the compound acting at the alpha7 nicotinic acid receptor and the acetylcholinesterase inhibitor are administered at very low, side-effect reducing or side-effect avoiding doses. In some embodiments the combination of agents provides a therapeutic benefit despite the fact that each of the two agents is administered at a dose that does not provide a therapeutic benefit when administered in the absence of the other agent. Moreover, the combination of agents may provide a benefit for a wider range or patients and/or over a longer period of treatment than is achieved when either is administered on its own.

(R)-7-chloro-N-(quinuclidin-3-yl)benzo[b]thiophene-2-carboxamide and pharmaceutically acceptable salts thereof (e.g., (R)-7-chloro-N-(quinuclidin-3-yl)benzo[b]thiophene-2-carboxamide hydrochloride monohydrate; depicted below) can be administered at a daily oral dose of (or no more than): 16 mg, 15 mg, 14 mg, 13 mg, 12 mg, 11 mg, 10 mg, 9 mg, 8.5 mg, 8.0 mg, 7.5 mg, 7 mg, 6.5 mg, 6 mg, 5.5 mg, 5 mg, 4.5 mg, 4 mg, 3 mg, 2.75 mg, 2.50 mg, 2.25 mg, 2 mg, 1.75 mg, 1.50 mg, 1.25 mg, 1 mg, 0.75 mg, 0.5 mg, 0.3 mg or even 0.1 mg. Thus, for example, it can be administered at a daily oral dose of 0.05-1.5 mg, 1.5-3 mg, 3-5 mg, 5-7 mg, 7-9 mg, 9-11 mg, 11-12 mg, 12-14 mg, or 14-16 mg.

(R)-7-(2-methoxyphenyl)-N-(quinuclidin-3-yl)benzofuran-2-carboxamide and pharmaceutically acceptable salts thereof can be administered at a daily oral dose of (or no more than): 16 mg, 15 mg, 14 mg, 13 mg, 12 mg, 11 mg, 10 mg, 9 mg, 8.5 mg, 8.0 mg, 7.5 mg, 7 mg, 6.5 mg, 6 mg, 5.5 mg, 5 mg, 4.5 mg, 4 mg, 3 mg, 2.75 mg, 2.50 mg, 2.25 mg, 2 mg, 1.75 mg, 1.50 mg, 1.25 mg, 1 mg, 0.75 mg, 0.5 mg, 0.3 mg or even 0.1 mg. Thus, for example, it can be administered at a daily oral dose of 0.05-1.5 mg, 1.5-3 mg, 3-5 mg, 5-7 mg, 7-9 mg, 9-11 mg, 11-12 mg, 12-14 mg, or 14-16 mg.

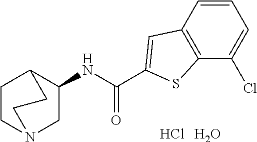 [SMILES: O=C(N[C@H]1CN2CCC1CC2)c1cc2cccc(Cl)c2s1 ; Cl ; O]

### (R)-7-chloro-N-(quinuclidin-3-yl)benzo[b]thiophene-2-carboxamide hydrochloride monohydrate

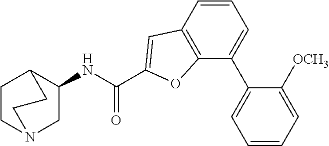 [SMILES: COc1ccccc1-c1cccc2cc(C(=O)N[C@H]3CN4CCC3CC4)oc12]

### (R)-7-(2-methoxyphenyl)-N-(quinuclidin-3-yl)benzofuran-2-carboxamide

In some cases the alpha 7 nicotinic receptor agonist is a compound of Formula I:

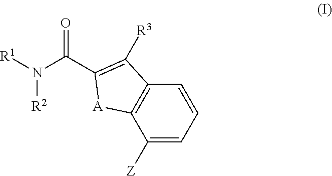

in which
R¹ represents 1-azabicyclo[2.2.2]oct-3-yl,
R² represents hydrogen or C1-C6-alkyl,
R³ represents hydrogen, halogen or C1-C6-alkyl,
A represents oxygen or sulfur, and
Z represents halogen, formyl, carbamoyl, cyano, trifluoromethyl, trifluoromethoxy, nitro, amino, formamido, acetamido, C1-C6-alkyl, C1-C6-alkyoxy, C1-C6-alkylthio, C1-C6-alkylamino, heteroaryl-carbonylamino, arylcarbonylamino, C1-C4-alkylsulfonylamino, di(arylsulfonyl)amino, C3-C6-cycloalkylcarbonylmethyl or amino(hydroxyimino)methyl, —O—C6 aryl optionally substituted with methyl, or C6 aryl substituted with one or more substituents selected from: halogen, cyano, methyl, —OCH₃, —CF₃, —OCF₃.

In some cases, R² is H, Z=CN, A=O, and R³ is H. In other cases, the compound is [(3R)-1-azabicyclo[2.2.2]oct-3-yl]-7-[2-(methoxy)phenyl]-1-benzofuran-2-carboxamide (Boess et al. 2007 J Pharm Exp Therap 321:716).

In some cases R² is hydrogen, R³ is hydrogen, A is sulfur, and Z represents halogen, formyl, carbamoyl, cyano, trifluoromethyl, methoxy, trifluoromethoxy, nitro, amino, formamido, acetamido, C1-C6-alkyl, C1-C6-alkyoxy, C1-C6-alkylthio, C1-C6-alkylamino, heteroaryl-carbonylamino, arylcarbonylamino, C1-C4-alkylsulfonylamino, di(arylsulfonyl)amino, C3-C6-cycloalkylcarbonylmethyl or amino(hydroxyimino)methyl (particularly halogen, cyano, trifluoromethyl, trifluoromethoxy, methyl, ethyl, methoxy, and ethoxy).

In some cases R² is H, Z=phenyl (optionally substituted with one or with one or more substituents selected from: halogen, cyano, methyl, —OCH₃, —CF₃, —OCF₃).

Compounds of Formula I and pharmaceutically acceptable salts thereof can be administered at a daily oral dose of (or no more than): 16 mg, 15 mg, 14 mg, 13 mg, 12 mg, 11 mg, 10 mg, 9 mg, 8.5 mg, 8.0 mg, 7.5 mg, 7 mg, 6.5 mg, 6 mg, 5.5 mg, 5 mg, 4.5 mg, 4 mg, 3 mg, 2.75 mg, 2.50 mg, 2.25 mg, 2 mg, 1.75 mg, 1.50 mg, 1.25 mg, 1 mg, 0.75 mg, 0.5 mg, 0.3 mg or even 0.1 mg. Thus, for example, it can be administered at a daily oral dose of 0.05-1.5 mg, 1.5-3 mg, 3-5 mg, 5-7 mg, 7-9 mg, 9-11 mg, 11-12 mg, 12-14 mg, or 14-16 mg.

For donepezil, the daily oral dose (or oral dose equivalent) used with (R)-7-chloro-N-(quinuclidin-3-yl)benzo[b]thiophene-2-carboxamide or pharmaceutically acceptable salt thereof can be 16 mg, 15 mg, 14 mg, 13 mg, 12 mg, 11 mg, 10 mg, 9 mg, 8 mg, 7 mg, 6 mg, 5 mg, 4.5 mg, 4 mg, 3.5 mg, 3 mg, 2.5 mg, 2 mg, 1 mg or 0.5 mg. The daily oral dose can be between 15 and 0.5 mg (e.g., 15-10 mg/day, 10-5 mg/day, 4.5-1.0 mg/day, 4.5-2.0 mg/day, 4.0-2.0 or 2.5 mg/day).

For rivastigmine the daily oral dose (or oral dose equivalent) for use in combination with (R)-7-chloro-N-(quinuclidin-3-yl)benzo[b]thiophene-2-carboxamide or pharmaceutically acceptable salt thereof or another alpha7 nicotinic receptor agonist described herein can be 16 mg, 15 mg, 14 mg, 13 mg, 12 mg, 11 mg, 10 mg, 9 mg, 8 mg, 7 mg, 6 mg, 5 mg, 4 mg, 3 mg, 2 mg, 1 mg, or 0.5 mg (e.g., 15-5 mg/day, 12-5 mg/day, 10-5 mg/day, 8-0.5 mg/day, 6-0.5 mg/day, 5-0.5 mg/day, 3-0.5 mg/day or 2-0.5 mg/day).

For galantamine the daily oral dose (or oral dose equivalent) for use in combination with (R)-7-chloro-N-(quinuclidin-3-yl)benzo[b]thiophene-2-carboxamide or pharmaceutically acceptable salt thereof can be 16 mg or less (e.g., 15 mg, 14 mg, 13 mg, 12 mg, 11 mg, 10 mg, 9 mg, 8 mg, 7 mg, 6 mg, 5 mg, 4 mg, 3 mg, 2 mg, 1 mg).

For tacrine the daily oral dose (or oral dose equivalent) for use in combination with (R)-7-chloro-N-(quinuclidin-3-yl)benzo[b]thiophene-2-carboxamide or pharmaceutically acceptable salt thereof can be 160 mg, 150 mg, 140 mg, 130 mg, 120 mg, 110 mg, 100 mg, 90 mg, 80 mg, 70 mg, 60 mg, 50 mg, 40 mg, 30 mg, 20 mg, 10 mg, 8 mg or less (e.g., 7-0.5 mg/day, 6-0.6 mg/day, 5-0.5 mg/day, 4-0.5 mg/day, 3-0.5 mg/day, 2-0.5 mg/day or 1-0.5 mg/day).

The table below illustrates some of the possible combinations for dosages of an alpha7 nicotinic receptor agonist (mg/day) (e.g., (R)-7-chloro-N-(quinuclidin-3-yl)benzo[b]thiophene-2-carboxamide or a pharmaceutically acceptable salt thereof, (R)-7-(2-methoxyphenyl)-N-(quinuclidin-3-yl)benzofuran-2-carboxamide or a pharmaceutically acceptable salt thereof, or a compound of Formula I) and acetylcholinesterse inhibitor (mg/day). In this table each “x” indicates a combination of doses (e.g., 11-9 mg/day of donepezil combined with 11-9 mg/day of an alpha 7 nicotinic receptor agonist).

| Dose of acetylcholinesterse inhibitor (mg/day) | | | | Dose of alpha7 nicotinic receptor agonist (mg/day) | | | | | | | |
|---|---|---|---|---|---|---|---|---|---|---|---|
| donepezil | rivastigmine | galantamine | tacrine | 16-15 | 15-13 | 13-11 | 11-9 | 9-7 | 7-5 | 5-3 | 3-1 |
| 16-15 | 16-15 | 16-15 | 150-130 | x | x | x | x | X | X | X | X |
| 15-13 | 15-13 | 15-13 | 130-110 | x | x | x | x | X | X | X | X |
| 13-11 | 13-11 | 13-11 | 110-90 | x | x | x | x | X | X | X | X |
| 11-9 | 11-9 | 11-9 | 90-70 | x | x | x | x | X | X | X | X |
| 9-7 | 9-7 | 9-7 | 70-50 | x | x | x | x | X | X | X | X |
| 7-5 | 7-5 | 7-5 | 50-30 | x | x | x | x | X | X | X | X |
| 5-3 | 5-3 | 5-3 | 30-10 | x | x | x | x | X | X | X | X |
| 3-1 | 3-1 | 3-1 | 15-13 | x | x | x | x | X | X | X | X |

A crystalline form of (R)-7-chloro-N-(quinuclidin-3-yl)benzo[b]thiophene-2-carboxamide hydrochloride monohydrate can be used, for example, Form I or Form II as described in PCT/US2011/036844.

Form I of (R)-7-chloro-N-(quinuclidin-3-yl)benzo[b]thiophene-2-carboxamide hydrochloride monohydrate can be characterized by an x-ray powder diffraction pattern having peaks expressed as 20 at: one or both of 17.48 and 20.58±0.20 degrees when measured against an internal silicon standard; at: one or both of 17.48 and 20.58±0.20 degrees when measured against an internal silicon standard and at one or more of 4.50, 9.04, 14.60, 15.14, 15.80, 16.60, 18.16, 18.44, 19.48, 21.74, and 25.46±0.20 degrees when measured against an internal silicon standard; one or both of 17.48 and 20.58±0.20 degrees when measured against an internal silicon standard and at two or more of 4.50, 9.04, 14.60, 15.14, 15.80, 16.60, 18.16, 18.44, 19.48, 21.74, and 25.46±0.20 degrees when measured against an internal silicon standard; one or both of 17.48 and 20.58±0.20 degrees when measured against an internal silicon standard and at four or more of 4.50, 9.04, 14.60, 15.14, 15.80, 16.60, 18.16, 18.44, 19.48, 21.74, and 25.46±0.20 degrees when measured against an internal silicon standard; or one or both of 17.48 and 20.58±0.20 degrees when measured against an internal silicon standard and at six or more of 4.50, 9.04, 14.60, 15.14, 15.80, 16.60, 18.16, 18.44, 19.48, 21.74, and 25.46±0.20 degrees when measured against an internal silicon standard; or one or both of 17.48 and 20.58±0.20 degrees when measured against an internal silicon standard and at eight or more of 4.50, 9.04, 14.60, 15.14, 15.80, 16.60, 18.16, 18.44, 19.48, 21.74, and 25.46±0.20 degrees when measured against an internal silicon standard.

Form II of (R)-7-chloro-N-(quinuclidin-3-yl)benzo[b]thiophene-2-carboxamide hydrochloride monohydrate can be characterized by an x-ray powder diffraction pattern having peaks expressed as 20 at: one or both of 21.16 and 21.38±0.20 degrees when measured against an internal silicon standard; one or both of 21.16 and 21.38±0.20 degrees when measured against an internal silicon standard and at one or more of 4.48, 9.00, 13.58, 15.62, 16.48, 19.02, 19.44, 22.46 and 25.00±0.20 degrees when measured against an internal silicon standard; one or both of 21.16 and 21.38±0.20 degrees when measured against an internal silicon standard and at two or more of 4.48, 9.00, 13.58, 15.62, 16.48, 19.02, 19.44, 22.46 and 25.00±0.20 degrees when measured against an internal silicon standard; one or both of 21.16 and 21.38±0.20 degrees when measured against an internal silicon standard and at four or more of 4.48, 9.00, 13.58, 15.62, 16.48, 19.02, 19.44, 22.46 and 25.00±0.20 degrees when measured against an internal silicon standard; one or both of 21.16 and 21.38±0.20 degrees when measured against an internal silicon standard and at six or more of 4.48, 9.00, 13.58, 15.62, 16.48, 19.02, 19.44, 22.46 and 25.00±0.20 degrees when measured against an internal silicon standard; or one or both of 21.16 and 21.38±0.20 degrees when measured against an internal silicon standard and at eight or more of 4.48, 9.00, 13.58, 15.62, 16.48, 19.02, 19.44, 22.46 and 25.00±0.20 degrees when measured against an internal silicon standard.

In some cases the acetylcholinesterase inhibitor can be administered by another route, e.g., transdermally.

Described below are methods for preparing crystalline Form I and crystalline Form II of (R)-7-chloro-N-(quinuclidin-3-yl)benzo[b]thiophene-2-carboxamide hydrochloride monohydrate. In the methods described below Form I is used as the starting form, but other forms can be used in place of Form I.

### Crystalline Form I of (R)-7-chloro-N-(quinuclidin-3-yl)benzo[b]thiophene-2-carboxamide hydrochloride monohydrate

Acetonitrile (90 mL) and water (10 mL) were mixed at room temperature. 1.0 ml of this solution was added to 100.7 mg of the crystalline Form I of (R)-7-chloro-N-(quinuclidin-3-yl)benzo[b]thiophene-2-carboxamide hydrochloride monohydrate. This suspension was stirred at 80° C. until solid component was dissolved, then the temperature was decreased to 40° C. for 80 minutes. During the cooling, spontaneous crystallization was observed round 52° C. To the suspension, 2.40 ml of acetonitrile was dropped slowly, and then the temperature was decreased to 10° C. for 60 minutes. The suspension was stirred at same temperature for 15 hours, and then the solid was filtered and washed with 0.20 ml of acetonitrile. After vacuum drying, 81.1 mg of the crystalline Form I was recovered.

### Crystalline Form II of (R)-7-chloro-N-(quinuclidin-3-yl)benzo[b]thiophene-2-carboxamide hydrochloride monohydrate

Acetonitrile (90 mL) and water (10 mL) were mixed at room temperature. 1.0 ml of this solution was added to 100.9 mg of the crystalline Form I of (R)-7-chloro-N-(quinuclidin-3-yl)benzo[b]thiophene-2-carboxamide hydrochloride monohydrate. This suspension was stirred at 80° C. until solid component was dissolved, then the temperature was decreased to 10° C. for 140 minutes. During the cooling, spontaneous crystallization was observed round 51° C. The suspension was stirred at same temperature for 15 hours, then the solid was filtered and washed with 0.20 ml of acetonitrile. After vacuum drying, 48.7 mg of the crystalline Form II was recovered.

For acetylcholinesterase inhibitors it is thought that for effectiveness in treating certain conditions, they must be administered so as to achieve a red blood cell acetylcholinesterase inhibition of at least 65%. In the some embodiments described herein the acetylcholinesterase inhibitor can be administered at a higher doses that achieves red blood cell acetylcholinesterase inhibition of greater than 65% or a lower dose that achieves red blood cell acetylcholinesterase inhibition of less than 65%, e.g., 55% (60-25%, 50-25%, 40-25% 50%, 45%, 40%, 35%, 30%, or 25% inhibition).

Described herein is a method for treating inflammation comprising administering to a patient: (a) (R)-7-chloro-N-(quinuclidin-3-yl)benzo[b]thiophene-2-carboxamide or a pharmaceutically acceptable salt thereof, (R)-7-(2-methoxyphenyl)-N-(quinuclidin-3-yl)benzofuran-2-carboxamide; or a compound of Formula I or a pharmaceutically acceptable salt thereof and (b) an acetylcholinesterase inhibitor. In various cases: the patient is suffering from or at risk of developing an inflammatory disorder, the acetylcholinesterase inhibitor is selected from tacrine, donepezil, rivastigmine and galantamine, the acetylcholinesterase inhibitor is selected from donepezil, rivastigmine and galantamine, the acetylcholinesterase inhibitor is selected from donepezil and rivastigmine, the patient has been administered an acetylcholinesterase inhibitor for a period of time (hours, days, weeks, months) prior to being administered (R)-7-chloro-N-(quinuclidin-3-yl)benzo[b]thiophene-2-carboxamide or a pharmaceutically acceptable salt thereof, (R)-7-(2-methoxyphenyl)-N-(quinuclidin-3-yl)benzofuran-2-carboxamide; or a compound of Formula I or a pharmaceutically acceptable salt thereof.

Also described is a method for treating inflammation comprising administering to a patient both: (a) (R)-7-chloro-N-(quinuclidin-3-yl)benzo[b]thiophene-2-carboxamide or a pharmaceutically acceptable salt thereof, (R)-7-(2-methoxyphenyl)-N-(quinuclidin-3-yl)benzofuran-2-carboxamide; or a compound of Formula I or a pharmaceutically acceptable salt thereof at a subclinical dose (e.g., a dose that does not reduce inflammation when administered by itself or in the absence of an acetylcholinestesterase inhibitor) and (b) an acetylcholinesterase inhibitor, also at a subclinical dose (a dose that does not reduce inflammation when administered by itself or in the absence of (R)-7-chloro-N-(quinuclidin-3-yl)benzo[b]thiophene-2-carboxamide or a pharmaceutically acceptable salt thereof) e.g., a dose that achieves red blood cell acetylcholinesterase inhibition of less than 60%, 50%, 40% or 30%.

Also described is a pharmaceutical composition comprising ((R)-7-chloro-N-(quinuclidin-3-yl)benzo[b]thiophene-2-carboxamide or a pharmaceutically acceptable salt thereof, (R)-7-(2-methoxyphenyl)-N-(quinuclidin-3-yl)benzofuran-2-carboxamide; or a compound of Formula I or a pharmaceutically acceptable salt thereof and an acetylcholinesterase inhibitor. In various cases: the acetylcholinesterase inhibitor is selected from tacrine, donepezil, rivastigmine and galantamine; the acetylcholinesterase inhibitor is selected from donepezil, rivastigmine and galantamine; the acetylcholinesterase inhibitor is selected from donepezil and rivastigmine; and the acetylcholinesterase inhibitor is donepezil.

The methods can be used to treat inflammation associated with variety of conditions, including: appendicitis, ulcers, peritonitis, pancreatitis, acute or ischemic colitis, diverticulitis, hepatitis, Crohn's disease, enteritis, ischemia/reperfusion injury, sepsis, septicemia, endotoxic shock, cachexia, urethritis, bronchitis, emphysema, rhinitis, cystic fibrosis, rheumatoid arthritis, synovitis, myasthenia gravis, thryoiditis, systemic lupus erythematosus, allograft rejection, and graft-versus-host disease. In other cases the disorder is selected from: appendicitis, peptic, gastric or duodenal ulcers, peritonitis, pancreatitis, acute or ischemic colitis, diverticulitis, epiglottitis, achalasia, cholangitis, cholecystitis, hepatitis, Crohn's disease, enteritis, Whipple's disease, asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, pneumoultramicroscopic silicovolcanoconiosis, alvealitis, bronchiolitis, pharyngitis, pleurisy, sinusitis, influenza, respiratory syncytial virus, herpes, disseminated bacteremia, Dengue fever, candidiasis, malaria, filariasis, amebiasis, hydatid cysts, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thryoiditis, systemic lupus erythematosus, Goodpasture's syndrome, Behcet's syndrome, allograft rejection, graft-versus-host disease, Type I diabetes, ankylosing spondylitis, Berger's disease, Type II diabetes, Berger's disease, Retier's syndrome, or Hodgkins disease. The method can also be used to treat inflammation within the central nervous system. In certain cases, the inflammation is selected from: sepsis, inflammation associated with sepsis septicemia, inflammation associated with endotoxic shock. In some cases, the methods can be used to treat inflammation following surgery.

The route of administration for the alpha 7 receptor agonist is preferably oral, but can vary with the condition to be treated. Thus, depending on the condition, the agents can be administered orally, parenterally, intranasally, vaginally, rectally, lingually, sublingually, bucally, intrabuccaly and transdermally to the patient. It is not necessary for both agents to be administered by the same route.

Also described is a daily unit dosage pharmaceutical composition comprising (R)-7-chloro-N-(quinuclidin-3-yl)benzo[b]thiophene-2-carboxamide or a pharmaceutically acceptable salt thereof, (R)-7-(2-methoxyphenyl)-N-(quinuclidin-3-yl)benzofuran-2-carboxamide; or a compound of Formula I or a pharmaceutically acceptable salt thereof and an acetylcholinesterase inhibitor and a pharmaceutically acceptable carrier.

Also described is a packaged pharmaceuticals comprising a package containing a first unit dosage of a pharmaceutical composition comprising: (a) (R)-7-chloro-N-(quinuclidin-3-yl)benzo[b]thiophene-2-carboxamide or a pharmaceutically acceptable salt thereof, (R)-7-(2-methoxyphenyl)-N-(quinuclidin-3-yl)benzofuran-2-carboxamide, or a compound of Formula I or a pharmaceutically acceptable salt thereof and (b) a second unit dosage pharmaceutical composition comprising an acetylcholinesterase inhibitor.

In some embodiments, R² is H, Z=CN, A=O, and R³ is H. In other embodiments, the compound is [(3R)-1-azabicyclo[2.2.2]oct-3-yl]-7-[2-(methoxy)phenyl]-1-benzofuran-2-carboxamide (Boers et al. 2007 J Pharm Exp Therap 321:716).

In another aspect, the patient is treated with a compound of Formula I wherein R² is hydrogen, R³ is hydrogen, A is sulfur, and Z represents halogen, formyl, carbamoyl, cyano, trifluoromethyl, methoxy, trifluoromethoxy, nitro, amino, formamido, acetamido, C1-C6-alkyl, C1-C6-alkyoxy, C1-C6-alkylthio, C1-C6-alkylamino, heteroaryl-carbonylamino, arylcarbonylamino, C1-C4-alkylsulfonylamino, di(arylsulfonyl)amino, C3-C6-cycloalkylcarbonylmethyl or amino(hydroxyimino)methyl (particularly halogen, cyano, trifluoromethyl, trifluoromethoxy, methyl, ethyl, methoxy, and ethoxy).

In certain cases the compound acting at the alpha7 nicotinic receptor is administered at a dosage that results in free plasma concentration of the compound in a human patient that is 5-, 10-, 15-, 20-, 30, 40- or 50-fold or more below the EC50 for the compound at the human alpha7 receptor when expressed in ooyctes. Thus, in some instances the alpha 7 receptor agonist is administered at a dosage that, based on EC50 measurements would not be expected to be effective.

The treatment with the two agents can also be used to: reduce tissue damage associated with inflammation, reduce the risk of developing inflammation in a patient not suffering from inflammation by administering the agents before inflammation fully develops; or reduce the severity of inflammation developed in a patient by administering the agents before inflammation fully develops. The two agents can be administered at the same time either in the same composition or in two different compositions. In addition, the agents can be administered at different times.

Also described herein is a pharmaceutical composition comprising: (R)-7-chloro-N-(quinuclidin-3-yl)benzo[b]thiophene-2-carboxamide or a pharmaceutically acceptable salt thereof or [(3R)-1-azabicyclo[2.2.2]oct-3-yl]-7-[2-(methoxy)phenyl]-1-benzofuran-2-carboxamide or a pharmaceutically acceptable salt thereof and a acetylcholinesterase inhibitor (e.g., tacrine, donepezil, rivastigmine or galantamine) and a pharmaceutically acceptable carrier.

“Dose” is the amount of active pharmaceutical ingredient (API) administered to a patient. For example, a 1 mg daily dose means that 1 mg of API is administered to a patient each day.

In various embodiments described herein the preferred form of (R)-7-chloro-N-(quinuclidin-3-yl)benzo[b]thiophene-2-carboxamide is (R)-7-chloro-N-(quinuclidin-3-yl)benzo[b]thiophene-2-carboxamide hydrochloride monohydrate.

### BRIEF DESCRIPTION OF THE DRAWINGS

FIG. 1 depicts a protocol for administering acetylcholine and (R)-7-chloro-N-(quinuclidin-3-yl)benzo[b]thiophene-2-carboxamide hydrochloride monohydrate to Xenopus oocytes.

FIG. 2 depicts the results of an assay showing the action of acetylcholine and (R)-7-chloro-N-(quinuclidin-3-yl)benzo[b]thiophene-2-carboxamide hydrochloride monohydrate.

### DETAILED DESCRIPTION

### (R)-7-chloro-N-(quinuclidin-3-yl)benzo[b]thiophene-2-carboxamide hydrochloride monohydrate and acetylcholine have a synergistic effect even at very low doses of (R)-7-chloro-N-(quinuclidin-3-yl)benzo[b]thiophene-2-carboxamide hydrochloride monohydrate

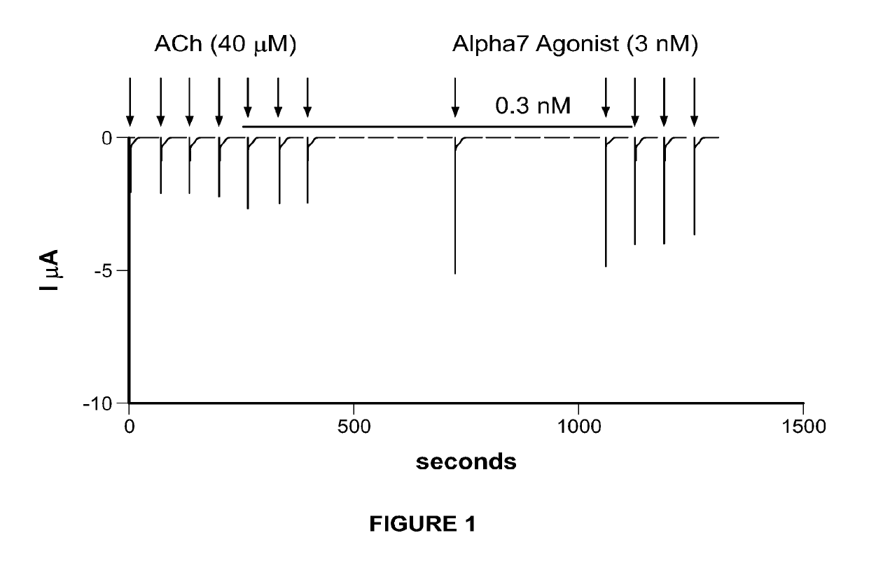

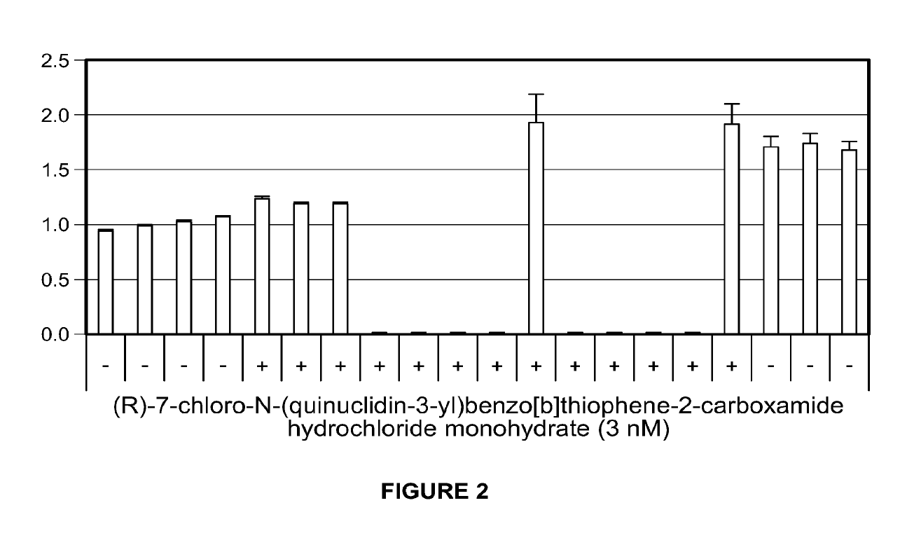

Studies in oocytes demonstrate that (R)-7-chloro-N-(quinuclidin-3-yl)benzo[b]thiophene-2-carboxamide is (R)-7-chloro-N-(quinuclidin-3-yl)benzo[b]thiophene-2-carboxamide hydrochloride monohydrate can have a synergistic effect when administered with acetylcholine even at every low concentrations of (R)-7-chloro-N-(quinuclidin-3-yl)benzo[b]thiophene-2-carboxamide is (R)-7-chloro-N-(quinuclidin-3-yl)benzo[b]thiophene-2-carboxamide hydrochloride monohydrate, i.e., well below the expected EC50. In the study described below currents were measures in Xenopus oocytes. Acetylcholine at 40 micromolar was administered prior to administration of (R)-7-chloro-N-(quinuclidin-3-yl)benzo[b]thiophene-2-carboxamide is (R)-7-chloro-N-(quinuclidin-3-yl)benzo[b]thiophene-2-carboxamide hydrochloride monohydrate at 0.3 nM using the protocol depicted in FIG. 1. The currents evoked (average of 3 cells) are shown in FIG. 2. Currents were normalized to unity versus the average acetylcholine-evoked current measured during the stabilization period (4 time points). Note the large current increase observed after 8 and 16 minutes incubation in presence of 0.3 nM (R)-7-chloro-N-(quinuclidin-3-yl)benzo[b]thiophene-2-carboxamide hydrochloride monohydrate.

### Assay for Alpha7 Receptor Agonism

The EC50 for an alpha7 receptor agonist can be measured using Xenopus oocytes as described previously (Boess et al. 2007 J Pharm Exp Therap 321:716; Methfessel et al., 1986 Pflueg Arch Eur J Physiol 407:577-588; Schnizler et at 2003 Receptors Channels 9:41-48). Briefly, pieces of ovary are excised from anesthetized adult female Xenopus laevi and treated with 2 mg/ml collagenase to release the oocytes from the follicle. Intact stage V oocytes are selected and placed into individual wells of 96-well plates filled with modified Barth's solution (88 mM NaCl, 1 mM KCl, 0.82 mM MgSO₄, 0.41 mM, Ca(NO₃)₂, 2.4 mM NaHCO₃, and 5 mM Tris-HCl, pH 7.4, with 50 g/ml gentamicin). Approximately 30 nl of cDNA solution, containing expression plasmids with inserts coding for the human alpha7 receptor is injected into the germinal vesicle of each oocyte using an automated system. Injected oocytes are incubated at 19° C. for 3 to 8 days in modified Barth's solution before the measurements are taken. For electrophysiological recording, oocytes are impaled with two glass microelectrodes filled with pipette solution (1.5 M potassium acetate and 0.1M KCl). Voltage clamp is performed with a standard voltage-clamp amplifier (Gene-Clamp 500 amplifier; Molecular Devices, Sunnyvale, Calif.). Oocytes are superfused with normal frog Ringer's solution (115 mM NaCl, 2.5 mM KCl, 1.8 mM CaCl₂, and 10 mM HEPES, pH 7.2). Solutions of test compounds in normal frog Ringer's solution are superfused through the recording chamber for 20 s while the voltage-clamp current is recorded. The washout time between applications of test solutions was 5 min. Each measurement of a test solution is preceded by the application of a standard concentration of acetylcholine (for nAChRs) or serotonin (for 5-HT3 receptors). Standard concentrations producing approximately 10 to 20% of the maximum response for each receptor can be used as internal controls for differences in expression levels between different oocytes and changes of the response during an experiment. Current amplitudes evoked by the test solutions are normalized to that of the preceding acetylcholine or serotonin application. For human alpha 7 nAChRs, 50 M acetylcholine can be used as reference standard. By testing with various concentration of the alpha7 receptor agonist the EC50 for agonism can be determined.

1. A method for treating inflammation comprising administering to a patient (R)-7-chloro-N-(quinuclidin-3-yl)benzo[b]thiophene-2-carboxamide or a pharmaceutically acceptable salt thereof or (R)-7-(2-methoxyphenyl)-N-(quinuclidin-3-yl)benzofuran-2-carboxamide or a pharmaceutically acceptable salt thereof and an acetylcholinesterase inhibitor.

2. A method for treating inflammation comprising administering to a patient a compound of Formula I, or a pharmaceutically acceptable salt thereof, and an acetylcholinesterase inhibitor 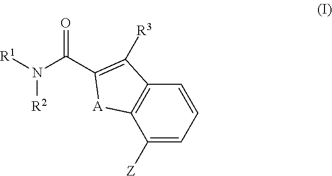
in which
R¹ represents 1-azabicyclo[2.2.2]oct-3-yl,
R² represents hydrogen or C1-C6-alkyl,
R³ represents hydrogen, halogen or C1-C6-alkyl,
A represents oxygen or sulfur, and
Z represents halogen, formyl, carbamoyl, cyano, trifluoromethyl, trifluoromethoxy, nitro, amino, formamido, acetamido, C1-C6-alkyl, C1-C6-alkyoxy, C1-C6-alkylthio, C1-C6-alkylamino, heteroaryl-carbonylamino, arylcarbonylamino, C1-C4-alkylsulfonylamino, di(arylsulfonyl)amino, C3-C6-cycloalkylcarbonylmethyl, amino(hydroxyimino)methyl, —O—C6 aryl optionally substituted with methyl, or C6 aryl optionally substituted with one or more substituents selected from: halogen, cyano, methyl, —OCH₃, —CF₃, —OCF₃.

3. The method of claim 2 wherein R² is hydrogen, R³ is hydrogen, A is sulfur, and Z represents halogen, formyl, carbamoyl, cyano, trifluoromethyl, trifluoromethoxy, nitro, amino, formamido, acetamido, C1-C6-alkyl, C1-C6-alkoxy, C1-C6-alkylthio, C1-C6-alkylamino, heteroaryl-carbonylamino, arylcarbonylamino, C1-C4-alkylsulfonylamino, di(arylsulfonyl)amino, C3-C6-cycloalkylcarbonylmethyl or amino(hydroxyimino)methyl.

4. The method of claim 3 wherein Z is selected from: halogen, cyano, trifluoromethyl, trifluoromethoxy, methyl, ethyl, methoxy, and ethoxy.

5. The method of claim 1, wherein the inflammation is sepsis or associated with appendicitis, ulcer, peritonitis, pancreatitis, acute or ischemic colitis, diverticulitis, hepatitis, Crohn's disease, enteritis, ischemia/reperfusion injury, sepsis, septicemia, endotoxic shock, cachexia, urethritis, bronchitis, emphysema, rhinitis, cystic fibrosis, rheumatoid arthritis, synovitis, myasthenia gravis, thryoiditis, systemic lupus erythematosus, allograft rejection, or graft-versus-host disease.

6. The method of claim 1, wherein the acetylcholinesterase inhibitor is selected from tacrine, donepezil, rivastigmine and galantamine.

7. The method of claim 1, comprising treating the patient with (R)-7-chloro-N-(quinuclidin-3-yl)benzo[b]thiophene-2-carboxamide hydrochloride and an acetylcholinesterase inhibitor.

8. The method of claim 1, comprising treating the patient with (R)-7-(2-methoxyphenyl)-N-(quinuclidin-3-yl)benzofuran-2-carboxamide or a pharmaceutically acceptable salt thereof and an acetylcholinesterase inhibitor.

9. The method of claim 7 wherein the (R)-7-chloro-N-(quinuclidin-3-yl)benzo[b]thiophene-2-carboxamide hydrochloride is crystalline Form I.

10. The method of claim 1 wherein the (R)-7-chloro-N-(quinuclidin-3-yl)benzo[b]thiophene-2-carboxamide or a pharmaceutically acceptable salt thereof or (R)-7-(2-methoxyphenyl)-N-(quinuclidin-3-yl)benzofuran-2-carboxamide or a pharmaceutically acceptable salt thereof is administered at a daily dosage selected from: 16 mg, 15 mg, 14 mg, 13 mg, 12 mg, 11 mg, 10 mg, 9 mg, 8.5 mg, 8.0 mg, 7.5 mg, 7 mg, 6.5 mg, 6 mg, 5.5 mg, 5 mg, 4.5 mg, 4 mg, 3 mg, 2.75 mg, 2.50 mg, 2.25 mg, 2 mg, 1.75 mg, 1.50 mg, 1.25 mg, 1 mg, 0.75 mg, 0.5 mg, 0.3 mg 13.

11. A pharmaceutical composition comprising (R)-7-chloro-N-(quinuclidin-3-yl)benzo[b]thiophene-2-carboxamide or a pharmaceutically acceptable salt thereof or (R)-7-(2-methoxyphenyl)-N-(quinuclidin-3-yl)benzofuran-2-carboxamide or a pharmaceutically acceptable salt thereof and an acetylcholinesterase inhibitor.

12. The pharmaceutical composition of claim 11 wherein acetylcholinesterase inhibitor is selected from tacrine, donepezil, rivastigmine and galantamine.
